(19) United States
(12) Patent Application Publication
(10) Pub. No.: US 2016/0176800 A1
(43) Pub. Date: Jun. 23, 2016

(54) Microalgal Derived Cleaners and Surfactants

(71) Applicant: Solazyme, Inc., South San Francisco, CA (US)

(72) Inventors: Celine Schiff-Deb, South San Francisco, CA (US); Timothy Dummer, South San Francisco, CA (US)

(21) Appl. No.: 14/973,072

(22) Filed: Dec. 17, 2015

Related U.S. Application Data
(60) Provisional application No. 62/093,406, filed on Dec. 17, 2014.

(51) Int. Cl. C07C 67/00; C11D 1/90; C11D 1/42; C11D 1/12; C11D 1/62

(52) U.S. Cl. CPC C07C 67/00; C11D 1/12; C11D 1/62; C11D 1/42; C11D 1/90

(57) ABSTRACT

Microalgal derived cleaners and surfactants, methods for their preparation, and compositions comprising the surfactants are provided.

### CROSS REFERENCE TO RELATED APPLICATION

This application claims the benefit under 35 USC 119(e) of U.S. Provisional Patent Application No. 62/093,406, filed Dec. 17, 2014 which is incorporated herein by reference in its entirety.

### BACKGROUND

Surfactants are surface active agents that reduce the surface tension between materials or a mixture of materials having dissimilar polarity, thereby increasing their compatibility and stability. For example oil, which is not normally compatible with water, can be solubilized in an aqueous solution through the use of surfactants. Surfactants are thus widely used in cleaners, detergents, food products, and in many other applications including personal care, home care, and industrial and institutional applications. Given the broad usage and demand for surfactants, concerns have been raised regarding the environmental impact of surfactants derived from petroleum or from palm oil or in the disposal of petroleum derived surfactants. Alternative surfactants that are derived from renewable and sustainable sources and that are biodegradable are desirable.

### SUMMARY

In one embodiment, the present invention provides microalgal derived surfactants, methods for their preparation, and compositions comprising the surfactants.

In one embodiment, provided is method for preparing a surfactant, the method comprising:
a) providing a triglyceride oil produced by a microalgae; and
b) subjecting the oil to one or more chemical reactions to produce a surfactant

In some embodiments, the surfactant is an anionic surfactant.

In other embodiments, the surfactant is a sulfonate having a —SO₃M moiety wherein M is an alkali metal.

In some embodiments, the sulfonate is an alkyl ester sulfonate of formula

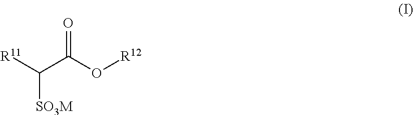

or a mixture thereof,
wherein R¹¹ is independently a C4-C16 alkyl group, R¹² is independently a C1-C5 alkyl group, and M is an alkali metal. In some embodiments, the alkyl ester sulfonate is a methyl ester sulfonate where R¹² is methyl.

In some embodiments, the sulfonate is a taurate of formula

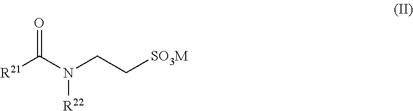

or a mixture thereof,
wherein R²¹ is independently a C5-C17 alkyl group or octadec-9-ene and R²² is independently H or a C1-C5 alkyl group. In some embodiments, the sulfonate is N-methyl alkyl taurate where R²² is methyl.

In some embodiments, the sulfonate is a sulfosuccinate of formula

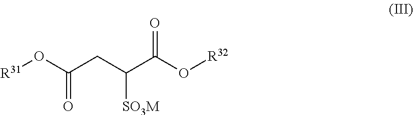

or a mixture thereof,
wherein R³¹ and R²² are independently a C6-C18 alkyl group or octadec-9-ene and M is an alkali metal.

In some embodiments, the sulfonate is an isethionate of formula

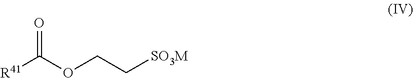

or a mixture thereof,
wherein R⁴¹ is independently a C6-C18 alkyl group or octadec-9-ene and M is an alkali metal.

In some embodiments, the surfactant is a sulfate having a —OSO₃M moiety wherein M is an alkali metal. In some embodiments, the sulfate is an alkyl sulfate of formula

R⁵¹OSO₃M (V)

or a mixture thereof,
wherein R⁵¹ is independently a C6-C18 alkyl group or octadec-9-ene and M is an alkali metal.

In some embodiments, the alkyl sulfate is a lauryl sulfate. In other embodiments, the alkyl sulfate is sodium lauryl sulfate.

In some embodiments, the sulfate is an alkyl ether sulfate.

In some embodiments, the alkyl ether sulfate is of formula R⁶¹(OCH₂CH₂)ₙOSO₃M (VI)

or a mixture thereof, wherein R⁶¹ is independently a C6-C18 alkyl group or octadec-9-ene, n is 1-50, and M is an alkali metal. In some embodiments, n is 2-7. In other embodiments, R⁶¹ is C12.

In some embodiments, the sulfate is of formula

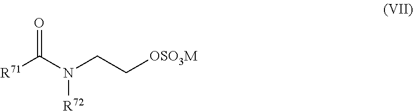

or a mixture thereof,
wherein R⁷¹ is independently a C5-C17 alkyl group or octadec-9-ene and R⁷² is independently H or a C1-C5 alkyl group. In some embodiments, R⁷² is methyl.

In some embodiments, the sulfate is a glyceride sulfate of formula

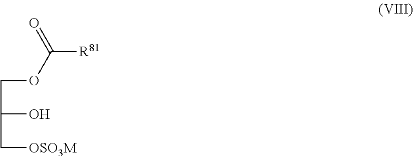

or a mixture thereof,
wherein R⁸¹ is independently a C6-C18 alkyl group or octadec-9-ene, and M is an alkali metal.

In some embodiments, the anionic surfactant comprises an amino acid moiety.

In some embodiments, the surfactant is

an N-acyl glutamate of formula

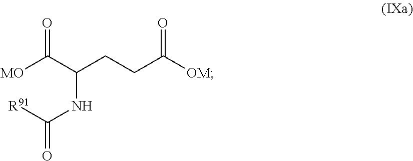

an N-acyl glycinate of formula

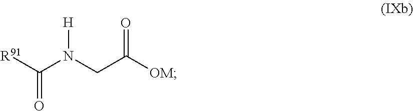

an N-acyl alaninate of formula

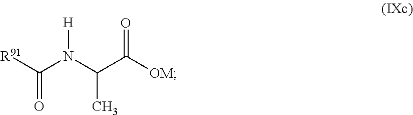

an N-acyl sarcosinate of formula

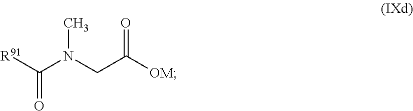

or a mixture thereof,
wherein R⁹¹ is independently a C6-C18 alkyl group or octadec-9-ene, and M is independently H or an alkali metal.

In some embodiments, the surfactant is an ether carboxylate of formula

R¹⁰¹(OCH₂CH₂)ₙOCH₂COOM (X)

or a mixture thereof,
wherein R¹⁰¹ is independently a C6-C18 alkyl group or octadec-9-ene, and M is independently H or an alkali metal.

In some embodiments, M is sodium.

In some embodiments, the surfactant is a nonionic surfactant.

In some embodiments, the nonionic surfactant comprises one or more of ethylene oxide or propylene oxide moieties or combinations thereof and an OH or SH moiety.

In some embodiments, the nonionic surfactant is of formula R¹¹¹(OCH₂CH₂)ₙAH (XI)

or a mixture thereof,
wherein R¹¹¹ is independently a C6-C18 alkyl group or octadec-9-ene, A is O or S, and n is an integer from 1-50. In some embodiments, n is 2-7.

In some embodiments, the nonionic surfactant is an alkyl polyglycoside.

In some embodiments, the nonionic surfactant comprises a glycerin moiety.

In some embodiments, the nonionic surfactant is of formula

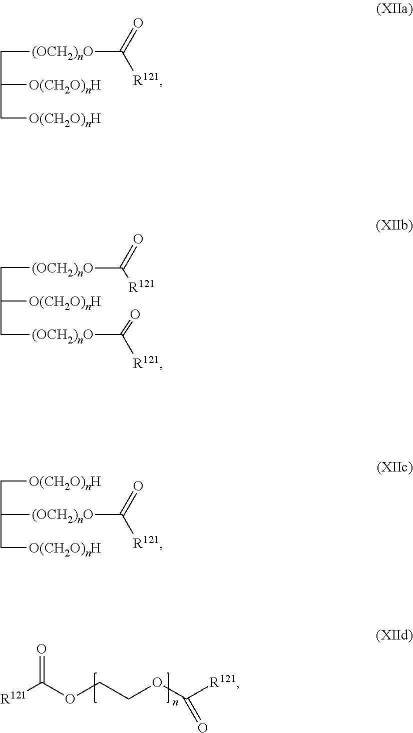

or a mixture thereof,
wherein R¹²¹ is independently a C6-C18 alkyl group or octadec-9-ene, and n is independently an integer from 1 to 50.

In some embodiments, the nonionic surfactant is a hydroxyalkyl amide.

In some embodiments, the hydroxy amide is of formula

R¹³¹(C═O)NR¹³²CH₂CH₂OH (XIII)

or a mixture thereof,
wherein R¹³¹ is independently a C6-C18 alkyl group or octadec-9-ene and R¹³² is independently H or —CH₂CH₂OH.

In some embodiments, the surfactant is an amphoteric surfactant.

In some embodiments, the amphoteric surfactant is a betaine.

In some embodiments, the betaine is an alkyl betaine salt of formula

R¹⁴¹N⁺(CH₃)₂CH₂X (XIV),

or a mixture thereof,
wherein R¹⁴¹ is independently a C6-C18 alkyl group or octadec-9-ene, X is COOM or SO₃M, and M is an alkali metal.

In some embodiments, the betaine is an amido betaine salt of formula

R¹⁵¹(C═O)NH(CH₂)₃N⁺(CH₃)₂CH₂COOM (XV),

or a mixture thereof,
wherein R¹⁵¹ is independently a C6-C18 alkyl group or octadec-9-ene and M is an alkali metal.

In some embodiments, R′⁵′ is a C10, C12, or C14 alkyl group or a mixture thereof.

In some embodiments, the betaine is cocoamidopropylbetaine.

In some embodiments, the amphoteric surfactant is an amine oxide.

In some embodiments, the surfactant is a cationic surfactant.

In some embodiments, the surfactant comprises a quaternary ammonium salt.

In some embodiments, the surfactant is an ester quat.

In some embodiments, the the ester quat is an ammonium salt of formula

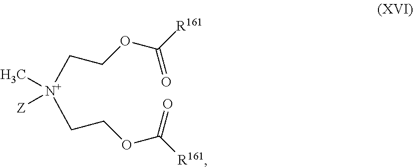

or a mixture thereof,
wherein R¹⁶¹ is independently a C6-C18 alkyl group or octadec-9-ene, and Z is —CH₃ or a C1-C6 hydroxyalkyl group. In some embodiments, Z is —CH₂CH₂OH. In other embodiments, Z is —CH₃.

In some embodiments, the microalgae is of the genus Prototheca, Auxenochlorella, Chlorella, or Parachlorella.

In some embodiments, the microalgae is of the species Prototheca moriformis.

In some embodiments, the microalgae is of the species Chlorella (Auxeochlorella) protothecoides.

In some embodiments, the microalgae is Chlorella (Auxeochlorella) protothecoides and the oil has a fatty acid profile of greater than 15% C16:0 and greater than 55% 18:1.

In some embodiments, the oil has a fatty acid profile of greater than 50%, 60%, 70%, or 80% combined C10:0 and C12:0.

In some embodiments, the oil has a fatty acid profile of greater than 60% C10:0 and C12:0 and greater than 10% C14:0.

In some embodiments, the oil has a fatty acid profile of greater than 40%, 45%, or 50% C14:0.

In some embodiments, the oil has a fatty acid profile of greater than 85% C18:1 and less than 3% polyunsaturates.

In some embodiments, the oil has a fatty acid profile of at least 70% SOS and no more than 4% trisaturates.

In some embodiments, the oil has a fatty acid profile of greater than 50% C18:0 and greater than 30% C18:1.

In some embodiments, provided is a composition comprising a surfactant disclosed herein.

In some embodiments, provided is a composition comprising C1-C4 esters of fatty acids derived from oil produced by a microalgae, the oil having a fatty acid profile of at least 35% C10:0 and 40% C12:0, and optionally less than 10% C14:0 fatty acids. In some cases, the oil has a fatty acid profile of at least 25% C10:0 and 35% C12:0. In some cases, the oil has a fatty acid profile of at least 25% C10:0 and 40% C12:0. In some embodiments, the ester is a methyl, ethyl, propyl, iso-propyl, butyl, or a tert-butyl ester.

In some embodiments, the surfactants provided herein are beneficial as antimicrobial agents. The surfactants can thus find use conferring antibacterial activity as part of the formulation of skin care products (e.g. creams, lotions, serums, cleansers, body butter), hair care products (e.g. shampoos, conditioners, masks, balms, leave-on oils), oral care products (e.g. toothpaste), color cosmetics (e.g. mascara, eye liner, foundation, lipsticks), lip care products (e.g. lip balm, lip gloss, lip gloss stain, lip scrub), and hand care and related products (e.g. hand cream, nail cream, dishwashing detergent).

In some embodiments, the surfactants are biocidal ester quats (quaternary ammonium compounds) for use as antimicrobials and disinfectants in household, industrial, institutional and personal care products. In some embodiments, the quats are derivatives of benzalkonium chloride, and didecyldimethylammonium chloride.

In some cases, the surfactants are prepared by subjecting fatty acids or fatty alcohols derived from the triglyceride oil produced by the microalgae to one or more chemical reactions.

In some embodiments, the composition is a cleaner, a metal working fluid, a pesticide, an agricultural adjuvant, a wetting agent, a dispersant, a buffer, a compatibility agent, a crop oil, a defoaming agent, a spreader, a detergent, a fabric softener, a cosmetic, a shampoo, a hair conditioner, a toothpaste, a sanitizer, an emulsion, an ink, an adhesive, a paint, a lubricant, a solvent, a coating, a sealant, an elastomer, a polymer, a pharmaceutical drug, a vaccine adjuvant, or a wellbore fluid.

In some embodiments, the metal working fluid is a metal forming, metal cutting, or a metal finishing fluid or a combination thereof.

In some embodiments, the wellbore fluid is a drilling fluid, a completion fluid, a work-over fluid, well stimulation fluid, or production fluid.

### DETAILED DESCRIPTION

### Definitions

“Microalgae” refers to eukaryotic microbial organisms that contain a chloroplast or other plastid, and optionally that are capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source. Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis. Examples of obligate heterotrophs include certain dinoflagellate algae species and species of the genus Prototheca. Microalgae include those belonging to the phylum Chlorophyta and in the class Trebouxiophyceae. Within this class are included microalgae belonging to the order Chlorellales, optionally the family Chlorellaceae, and optionally the genus Prototheca, Auxenochlorella, Chlorella, or Parachlorella.

“Microalgal oils” or “cell oils” refer to lipid components produced by microalgal cells such as triglycerides.

“Fatty acid profile” refers to the distribution of different carbon chain lengths and saturation levels of fatty acid moieties in a particular sample of biomass or oil. “Triglycerides” are lipids where three fatty acid moieties are attached to a glycerol moiety. A sample could contain lipids in which approximately 60% of the fatty acid moieties is C18:1, 20% is C18:0, 15% is C16:0, and 5% is C14:0. In cases in which a carbon length is referenced generically, such as “C18”, such reference can include any amount of saturation; for example, microalgal biomass that contains 20% lipid as C18 can include C18:0, C18:1, C18:2, and the like, in equal or varying amounts, the sum of which constitute 20% of the biomass.

“Cleaners” or “solvents” refers to substances and products used: (a) to wash, de-grease, clean, disinfect, buff, polish, shine or protect (i) buildings and facilities (including, without limitation, homes, factories, offices, hotels, convention centers, hospitals and medical facilities, schools and educational facilities, shops, restaurants, places of business, government and military facilities, warehouses and storage facilities, public and private utilities, oil and gas production rigs, parks and recreation facilities), (ii) hard surfaces (including, without limitation, floors, walls, ceilings, doors, windows, counters, furniture, tables, chairs, kitchen and appliance surfaces, laboratory surfaces and toilets), (iii) human or animal skin, and (iv) automotive or other transportation interior or exterior surfaces; or (b) as fuel system additives. These cleaners include those commonly characterized in the cleaning industry as Home, Industrial & Institutional Cleaners.

“Surfactants,” unless otherwise specified, include but are not limited to: “anionic surfactants” such as acyl isethionate, acyl sarcosinate, methyl esters of alpha-sulfo fatty acids (MES), alcohol sulfate (AS), alcohol ether sulfates (AES), and alkyl glyceryl ether (AGES); “nonionic surfactants” such as alcohol ethoxylates, alkyl glucosamides (AGA), alkyl polyglycosides (APG), amine oxides, ethoxylated/propoxylated types, fatty acid esters/fatty acid ethoxylates, and fatty alkanolamides; and “amphoteric surfactants” such as alkylamido betaines, alkyl betaines, amphoacetates, amphopropionate, phosphobetaines, and sulfobetaines.

### Microalgal Cells and Extracts

The microalgal cells can be prepared and heterotrophically cultured according to methods such as those described in WO2008/151149, WO2010/063031, WO2010/045368, WO2010/063032, WO2011/150411, WO2013/158938, 61/923,327 filed Jan. 3, 2014, PCT/US2014/037898 filed May 13, 2014, and in U.S. Pat. No. 8,557,249. The microalgal cells can be wild type cells or can be modified by genetic engineering and/or classical mutagenesis to alter their fatty acid profile and/or lipid productivity or other physical properties such as color.

In particular embodiments, the wild-type or genetically engineered microalgae comprise cells that are at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% or more oil by dry weight. Preferred organisms grow heterotrophically (on sugars in the absence of light).

In some embodiments, the microalgae is from the genus Chlorella. Chlorella is a genus of single-celled green algae, belonging to the phylum Chlorophyta. Chlorella cells are generally spherical in shape, about 2 to 10 μm in diameter, and lack flagella. Some species of Chlorella are naturally heterotrophic. In some embodiments, the microalgae used in the methods of the invention is Chlorella (auexnochlorella) protothecoides, Chlorella ellipsoidea, Chlorella minutissima, Chlorella zofinienesi, Chlorella luteoviridis, Chlorella kessleri, Chlorella sorokiniana, Chlorella fusca var. vacuolata Chlorella sp., Chlorella cf. minutissima or Chlorella emersonii. Other species of Chlorella those selected from the group consisting of anitrata, Antarctica, aureoviridis, candida, capsulate, desiccate, ellipsoidea (including strain CCAP 211/42), emersonii, fusca (including var. vacuolata), glucotropha, infusionum (including var. actophila and var. auxenophila), kessleri (including any of UTEX strains 397,2229,398), lobophora (including strain SAG 37.88), luteoviridis (including strain SAG 2203 and var. aureoviridis and lutescens), miniata, cf. minutissima, minutissima (including UTEX strain 2341), mutabilis, nocturna, ovalis, parva, photophila, pringsheimii, protothecoides (including any of UTEX strains 1806, 411, 264, 256, 255, 250, 249, 31, 29, 25 or CCAP 211/8D, or CCAP 211/17 and var. acidicola), regularis (including var. minima, and umbricata), reisiglii (including strain CCP 11/8), saccharophila (including strain CCAP 211/31, CCAP 211/32 and var. ellipsoidea), salina, simplex, sorokiniana (including strain SAG 211.40B), sp. (including UTEX strain 2068 and CCAP 211/92), sphaerica, stigmatophora, trebouxioides, vanniellii, vulgaris (including strains CCAP 211/11K, CCAP 211/80 and f. tertia and var. autotrophica, viridis, vulgaris, vulgaris f. tertia, vulgaris f. viridis), xanthella, and zofingiensis.

In addition to Chlorella, other genera of microalgae can also be used in the methods and compositions provided herein. In some embodiments, the microalgae is a species selected from the group consisting Parachlorella kessleri, Parachlorella beijerinckii, Neochloris oleabundans, Bracteacoccus, including B. grandis, B. cinnabarinas, and B. aerius, Bracteococcus sp. or Scenedesmus rebescens. Other nonlimiting examples of microalgae species include those species from the group of species and genera consisting of Achnanthes orientalis; Agmenellum; Amphiprora hyaline; Amphora, including A. coffeiformis including A.c. linea, A.c. punctata, A.c. taylori, A.c. tenuis, A.c. delicatissima, A.c. delicatissima capitata; Anabaena; Ankistrodesmus, including A. falcatus; Boekelovia hooglandii; Borodinella; Botryococcus braunii, including B. sudeticus; Bracteoccocus, including B. aerius, B. grandis, B. cinnabarinas, B. minor, and B. medionucleatus; Carteria; Chaetoceros, including C. gracilis, C. muelleri, and C. muelleri subsalsum; Chlorococcum, including C. infusionum; Chlorogonium; Chroomonas; Chrysosphaera; Cricosphaera; Crypthecodinium cohnii; Cryptomonas; Cyclotella, including C. cryptica and C. meneghiniana; Dunaliella, including D. bardawil, D. bioculata, D. granulate, D. maritime, D. minuta, D. parva, D. peircei, D. primolecta, D. salina, D. terricola, D. tertiolecta, and D. viridis; Eremosphaera, including E. viridis; Ellipsoidon; Euglena; Franceia; Fragilaria, including F. crotonensis; Gleocapsa; Gloeothamnion; Hymenomonas; Isochrysis, including I. aff galbana and I. galbana; Lepocinclis; Micractinium (including UTEX LB 2614); Monoraphidium, including M. minutum; Monoraphidium; Nannochloris; Nannochloropsis, including N. salina; Navicula, including N. acceptata, N. biskanterae, N. pseudotenelloides, N. pelliculosa, and N. saprophila; Neochloris oleabundans; Nephrochloris; Nephroselmis; Nitschia communis; Nitzschia, including N. alexandrina, N. communis, N. dissipata, N. frustulum, N. hantzschiana, N. inconspicua, N. intermedia, N. microcephala, N. pusilla, N. pusilla elliptica, N. pusilla monoensis, and N. quadrangular; Ochromonas; Oocystis, including O. parva and O. pusilla; Oscillatoria, including O. limnetica and O. subbrevis; Parachlorella, including P. beijerinckii (including strain SAG 2046) and P. kessleri (including any of SAG strains 11.80, 14.82, 21.11H9); Pascheria, including P. acidophila; Pavlova; Phagus; Phormidium; Platymonas; Pleurochrysis, including P. carterae and P. dentate; Prototheca, including P. stagnora (including UTEX 327), P. portoricensis, and P. moriformis (including UTEX strains 1441,1435, 1436, 1437, 1439); Pseudochlorella aquatica; Pyramimonas; Pyrobotrys; Rhodococcus opacus; Sarcinoid chrysophyte; Scenedesmus, including S. armatus and S. rubescens; Schizochytrium; Spirogyra; Spirulina platensis; Stichococcus; Synechococcus; Tetraedron; Tetraselmis, including T. suecica; Thalassiosira weissflogii; and Viridiella fridericiana.

Microalgae are cultured in liquid media to propagate biomass. Microalgal species are grown in a medium containing a fixed carbon and/or fixed nitrogen source in the absence of light. Such growth is known as heterotrophic growth. For some species of microalgae, for example, heterotrophic growth for extended periods of time such as 10 to 15 or more days under limited nitrogen conditions results accumulation of high lipid content in cells.

Microalgal culture media typically contains components such as a fixed carbon source (discussed below), a fixed nitrogen source (such as protein, soybean meal, yeast extract, cornsteep liquor, ammonia (pure or in salt form), nitrate, or nitrate salt), trace elements (for example, zinc, boron, cobalt, copper, manganese, and molybdenum in, e.g., the respective forms of ZnCl₂, H₃BO₃, CoCl₂.6H₂O, CuCl₂.2H₂O, MnCl₂.4H₂O and (NH₄)₆Mo₇O₂₄.4H₂O), optionally a buffer for pH maintenance, and phosphate (a source of phosphorous; other phosphate salts can be used). Other components include salts such as sodium chloride, particularly for seawater microalgae.

In a particular example, a medium suitable for culturing Chlorella protothecoides comprises Proteose Medium. This medium is suitable for axenic cultures, and a 1 L volume of the medium (pH ˜6.8) can be prepared by addition of 1 g of proteose peptone to 1 liter of Bristol Medium. Bristol medium comprises 2.94 mM NaNO₃, 0.17 mM CaCl₂.2H₂O, 0.3 mM MgSO₄.7H₂O, 0.43 mM, 1.29 mM KH₂PO₄, and 1.43 mM NaCl in an aqueous solution. For 1.5% agar medium, 15 g of agar can be added to 1 L of the solution. The solution is covered and autoclaved, and then stored at a refrigerated temperature prior to use. Other methods for the growth and propagation of Chlorella protothecoides to high oil levels as a percentage of dry weight have been described (see for example Miao and Wu, J. Biotechnology, 2004, 11:85-93 and Miao and Wu, Biosource Technology (2006) 97:841-846 (demonstrating fermentation methods for obtaining 55% oil dry cell weight)). High oil algae can typically be generated by increasing the length of a fermentation while providing an excess of carbon source under nitrogen limitation.

Solid and liquid growth media are generally available from a wide variety of sources, and instructions for the preparation of particular media that is suitable for a wide variety of strains of microorganisms can be found, for example, online at a site maintained by the University of Texas at Austin for its culture collection of algae (UTEX). For example, various fresh water media include ½, ⅓, ⅕, 1×, ⅔, 2×CHEV Diatom Medium; 1:1 DYIII/PEA+Gr+; Ag Diatom Medium; Allen Medium; BG11-1 Medium; Bold 1NV and 3N Medium; Botryococcus Medium; Bristol Medium; Chu's Medium; CR1, CR1-S, and CR1+Diatom Medium; Cyanidium Medium; Cyanophycean Medium; Desmid Medium; DYIII Medium; Euglena Medium; HEPES Medium; J Medium; Malt Medium; MES Medium; Modified Bold 3N Medium; Modified COMBO Medium; N/20 Medium; Ochromonas Medium; P49 Medium; Polytomella Medium; Proteose Medium; Snow Algae Media; Soil Extract Medium; Soilwater: BAR, GR−, GR−/NH4, GR+, GR+/NH4, PEA, Peat, and VT Medium; Spirulina Medium; Tap Medium; Trebouxia Medium; Volvocacean Medium; Volvocacean-3N Medium; Volvox Medium; Volvox-Dextrose Medium; Waris Medium; and Waris+Soil Extract Medium. Various Salt Water Media include: 1%, 5%, and 1× F/2 Medium; ½, 1×, and 2× Erdschreiber's Medium; ½, ⅓, ¼, ⅕, 1×, 5/3, and 2× Soil+Seawater Medium; ¼ ERD; ⅔ Enriched Seawater Medium; 20% Allen+80% ERD; Artificial Seawater Medium; BG11-1+0.36% NaCl Medium; BG11-1+1% NaCl Medium; Bold 1NV:Erdshreiber (1:1) and (4:1); Bristol-NaCl Medium; Dasycladales Seawater Medium; ½ and 1× Enriched Seawater Medium, including ES/10, ES/2, and ES/4; F/2+NH4; LDM Medium; Modified 1× and 2×CHEV; Modified 2× CHEV+Soil; Modified Artificial Seawater Medium; Porphridium Medium; and SS Diatom Medium.

Other suitable media for use with the methods of the invention can be readily identified by consulting other organizations that maintain cultures of microorganisms, such as SAG, CCAP, or CCALA. SAG refers to the Culture Collection of Algae at the University of Gottingen (Gottingen, Germany), CCAP refers to the culture collection of algae and protozoa managed by the Scottish Association for Marine Science (Scotland, United Kingdom), and CCALA refers to the culture collection of algal laboratory at the Institute of Botany (T\check{r}ebo\check{n}, Czech Republic).

Microorganisms useful in accordance with the methods of the present disclosure are found in various locations and environments throughout the world. As a consequence of their isolation from other species and their resulting evolutionary divergence, the particular growth medium for optimal growth and generation of oil and/or lipid and/or protein from any particular species of microbe can be difficult or impossible to predict, but those of skill in the art can readily find appropriate media by routine testing in view of the disclosure herein. In some cases, certain strains of microorganisms may be unable to grow on a particular growth medium because of the presence of some inhibitory component or the absence of some essential nutritional requirement required by the particular strain of microorganism. The examples below provide exemplary methods of culturing various species of microalgae to accumulate high levels of lipid as a percentage of dry cell weight.

Suitable fixed carbon sources for use in the medium, include, for example, glucose, fructose, sucrose, galactose, xylose, mannose, rhamnose, arabinose, N-acetylglucosamine, glycerol, floridoside, glucuronic acid, and/or acetate.

High lipid biomass from microalgae is an advantageous material for inclusion in cosmetic products compared to low lipid biomass, because it allows for the addition of less microalgal biomass to incorporate the same amount of lipid into a cosmetic composition. Process conditions can be adjusted to increase the percentage weight of cells that is lipid. For example, in certain embodiments, a microalgae is cultured in the presence of a limiting concentration of one or more nutrients, such as, for example, nitrogen, phosphorous, or sulfur, while providing an excess of a fixed carbon source, such as glucose. Nitrogen limitation tends to increase microbial lipid yield over microbial lipid yield in a culture in which nitrogen is provided in excess. In particular embodiments, the increase in lipid yield is at least about 10%, 50%, 100%, 200%, or 500%. The microbe can be cultured in the presence of a limiting amount of a nutrient for a portion of the total culture period or for the entire period. In some embodiments, the nutrient concentration is cycled between a limiting concentration and a non-limiting concentration at least twice during the total culture period.

In a steady growth state, the cells accumulate oil but do not undergo cell division. In one embodiment of the invention, the growth state is maintained by continuing to provide all components of the original growth media to the cells with the exception of a fixed nitrogen source. Cultivating microalgal cells by feeding all nutrients originally provided to the cells except a fixed nitrogen source, such as through feeding the cells for an extended period of time, results in a higher percentage of lipid by dry cell weight.

In other embodiments, high lipid biomass is generated by feeding a fixed carbon source to the cells after all fixed nitrogen has been consumed for extended periods of time, such as at least one or two weeks. In some embodiments, cells are allowed to accumulate oil in the presence of a fixed carbon source and in the absence of a fixed nitrogen source for over 20 days. Microalgae grown using conditions described herein or otherwise known in the art can comprise at least about 20% lipid by dry weight, and often comprise 35%, 45%, 55%, 65%, and even 75% or more lipid by dry weight. Percentage of dry cell weight as lipid in microbial lipid production can therefore be improved by holding cells in a heterotrophic growth state in which they consume carbon and accumulate oil but do not undergo cell division.

Organic nitrogen sources have been used in microbial cultures since the early 1900s. The use of organic nitrogen sources, such as corn steep liquor was popularized with the production of penicillin from mold. Researchers found that the inclusion of corn steep liquor in the culture medium increased the growth of the microoranism and resulted in an increased yield in products (such as penicillin). An analysis of corn steep liquor determined that it was a rich source of nitrogen and also vitamins such as B-complex vitamins, riboflavin panthothenic acid, niacin, inositol and nutrient minerals such as calcium, iron, magnesium, phosphorus and potassium (Ligget and Koffler, Bacteriological Reviews (1948); 12(4): 297-311). Organic nitrogen sources, such as corn steep liquor, have been used in fermentation media for yeasts, bacteria, fungi and other microorganisms. Non-limiting examples of organic nitrogen sources are yeast extract, peptone, corn steep liquor and corn steep powder. Non-limiting examples of preferred inorganic nitrogen sources include, for example, and without limitation, (NH₄)₂SO₄ and NH₄OH. In one embodiment, the culture media for carrying out the invention contains only inorganic nitrogen sources. In another embodiment, the culture media for carrying out the invention contains only organic nitrogen sources. In yet another embodiment, the culture media for carrying out the invention contains a mixture of organic and inorganic nitrogen sources.

In the methods of the invention, a bioreactor or fermentor is used to culture microalgal cells through the various phases of their physiological cycle. As an example, an inoculum of lipid-producing microalgal cells is introduced into the medium; there is a lag period (lag phase) before the cells begin to propagate. Following the lag period, the propagation rate increases steadily and enters the log, or exponential, phase. The exponential phase is in turn followed by a slowing of propagation due to decreases in nutrients such as nitrogen, increases in toxic substances, and quorum sensing mechanisms. After this slowing, propagation stops, and the cells enter a stationary phase or steady growth state, depending on the particular environment provided to the cells. For obtaining protein rich biomass, the culture is typically harvested during or shortly after then end of the exponential phase. For obtaining lipid rich biomass, the culture is typically harvested well after then end of the exponential phase, which may be terminated early by allowing nitrogen or another key nutrient (other than carbon) to become depleted, forcing the cells to convert the carbon sources, present in excess, to lipid. Culture condition parameters can be manipulated to optimize total oil production, the combination of lipid species produced, and/or production of a specific oil.

Bioreactors offer many advantages for use in heterotrophic growth and propagation methods. As will be appreciated, provisions made to make light available to the cells in photosynthetic growth methods are unnecessary when using a fixed-carbon source in the heterotrophic growth and propagation methods described herein. To produce biomass for use in cosmetics, microalgae are preferably fermented in large quantities in liquid, such as in suspension cultures as an example. Bioreactors such as steel fermentors (5000 liter, 10,000 liter, 40,000 liter, and higher are used in various embodiments of the invention) can accommodate very large culture volumes. Bioreactors also typically allow for the control of culture conditions such as temperature, pH, oxygen tension, and carbon dioxide levels. For example, bioreactors are typically configurable, for example, using ports attached to tubing, to allow gaseous components, like oxygen or nitrogen, to be bubbled through a liquid culture.

Bioreactors can be configured to flow culture media though the bioreactor throughout the time period during which the microalgae reproduce and increase in number. In some embodiments, for example, media can be infused into the bioreactor after inoculation but before the cells reach a desired density. In other instances, a bioreactor is filled with culture media at the beginning of a culture, and no more culture media is infused after the culture is inoculated. In other words, the microalgal biomass is cultured in an aqueous medium for a period of time during which the microalgae reproduce and increase in number; however, quantities of aqueous culture medium are not flowed through the bioreactor throughout the time period. Thus in some embodiments, aqueous culture medium is not flowed through the bioreactor after inoculation.

Bioreactors equipped with devices such as spinning blades and impellers, rocking mechanisms, stir bars, means for pressurized gas infusion can be used to subject microalgal cultures to mixing. Mixing may be continuous or intermittent. For example, in some embodiments, a turbulent flow regime of gas entry and media entry is not maintained for reproduction of microalgae until a desired increase in number of said microalgae has been achieved.

As briefly mentioned above, bioreactors are often equipped with various ports that, for example, allow the gas content of the culture of microalgae to be manipulated. To illustrate, part of the volume of a bioreactor can be gas rather than liquid, and the gas inlets of the bioreactor to allow pumping of gases into the bioreactor. Gases that can be beneficially pumped into a bioreactor include air, air/CO₂ mixtures, noble gases, such as argon, and other gases. Bioreactors are typically equipped to enable the user to control the rate of entry of a gas into the bioreactor. As noted above, increasing gas flow into a bioreactor can be used to increase mixing of the culture.

Increased gas flow affects the turbidity of the culture as well. Turbulence can be achieved by placing a gas entry port below the level of the aqueous culture media so that gas entering the bioreactor bubbles to the surface of the culture. One or more gas exit ports allow gas to escape, thereby preventing pressure buildup in the bioreactor. Preferably a gas exit port leads to a “one-way” valve that prevents contaminating microorganisms from entering the bioreactor.

The specific examples of bioreactors, culture conditions, and heterotrophic growth and propagation methods described herein can be combined in any suitable manner to improve efficiencies of microbial growth and lipid and/or protein production.

Concentration of Microalgae after Fermentation

Microalgal cultures generated according to the methods described above yield microalgal biomass in fermentation media. To prepare the biomass for use as a cosmetic composition, the biomass is concentrated, or harvested, from the fermentation medium. At the point of harvesting the microalgal biomass from the fermentation medium, the biomass comprises predominantly intact cells suspended in an aqueous culture medium. To concentrate the biomass, a dewatering step is performed. Dewatering or concentrating refers to the separation of the biomass from fermentation broth or other liquid medium and so is solid-liquid separation. Thus, during dewatering, the culture medium is removed from the biomass (for example, by draining the fermentation broth through a filter that retains the biomass), or the biomass is otherwise removed from the culture medium. Common processes for dewatering include centrifugation, filtration, and the use of mechanical pressure. These processes can be used individually or in any combination.

Centrifugation involves the use of centrifugal force to separate mixtures. During centrifugation, the more dense components of the mixture migrate away from the axis of the centrifuge, while the less dense components of the mixture migrate towards the axis. By increasing the effective gravitational force (i.e., by increasing the centrifugation speed), more dense material, such as solids, separate from the less dense material, such as liquids, and so separate out according to density. Centrifugation of biomass and broth or other aqueous solution forms a concentrated paste comprising the microalgal cells. Centrifugation does not remove significant amounts of intracellular water. In fact, after centrifugation, there may still be a substantial amount of surface or free moisture in the biomass (e.g., upwards of 70%), so centrifugation is not considered to be a drying step.

Filtration can also be used for dewatering. One example of filtration that is suitable for the present invention is tangential flow filtration (TFF), also known as cross-flow filtration. Tangential flow filtration is a separation technique that uses membrane systems and flow force to separate solids from liquids. For an illustrative suitable filtration method, see Geresh, Carb. Polym. 50; 183-189 (2002), which describes the use of a MaxCell A/G Technologies 0.45 uM hollow fiber filter. Also see, for example, Millipore Pellicon® devices, used with 100 kD, 300 kD, 1000 kD (catalog number P2C01MC01), 0.1 uM (catalog number P2VVPPV01), 0.22 uM (catalog number P2GVPPV01), and 0.45 uM membranes (catalog number P2HVMPV01). The retentate preferably does not pass through the filter at a significant level, and the product in the retentate preferably does not adhere to the filter material. TFF can also be performed using hollow fiber filtration systems. Filters with a pore size of at least about 0.1 micrometer, for example about 0.12, 0.14, 0.16, 0.18, 0.2, 0.22, 0.45, or at least about 0.65 micrometers, are suitable. Preferred pore sizes of TFF allow solutes and debris in the fermentation broth to flow through, but not microbial cells.

Dewatering can also be affected with mechanical pressure directly applied to the biomass to separate the liquid fermentation broth from the microbial biomass sufficient to dewater the biomass but not to cause predominant lysis of cells. Mechanical pressure to dewater microbial biomass can be applied using, for example, a belt filter press. A belt filter press is a dewatering device that applies mechanical pressure to a slurry (e.g., microbial biomass taken directly from the fermentor or bioreactor) that is passed between the two tensioned belts through a serpentine of decreasing diameter rolls. The belt filter press can actually be divided into three zones: the gravity zone, where free draining water/liquid is drained by gravity through a porous belt; a wedge zone, where the solids are prepared for pressure application; and a pressure zone, where adjustable pressure is applied to the gravity drained solids.

After concentration, microalgal biomass can be processed, as described hereinbelow, to produce vacuum-packed cake, algal flakes, algal homogenate, algal powder, algal flour, or algal oil.

### Chemical Composition of Microalgal Biomass

The microalgal biomass generated by the culture methods described herein comprises microalgal oil and/or protein as well as other constituents generated by the microorganisms or incorporated by the microorganisms from the culture medium during fermentation.

Microalgal biomass with a high percentage of oil/lipid accumulation by dry weight has been generated using different methods of culture, including methods known in the art. Microalgal biomass with a higher percentage of accumulated oil/lipid is useful in accordance with the present invention. Chlorella vulgaris cultures with up to 56.6% lipid by dry cell weight (DCW) in stationary cultures grown under autotrophic conditions using high iron (Fe) concentrations have been described (Li et al., Bioresource Technology 99(11):4717-22 (2008). Nanochloropsis sp. and Chaetoceros calcitrans cultures with 60% lipid by DCW and 39.8% lipid by DCW, respectively, grown in a photobioreactor under nitrogen starvation conditions have also been described (Rodolfi et al., Biotechnology & Bioengineering (2008)). Parietochloris incise cultures with approximately 30% lipid by DCW when grown phototropically and under low nitrogen conditions have been described (Solovchenko et al., Journal of Applied Phycology 20:245-251 (2008). Chlorella protothecoides can produce up to 55% lipid by DCW when grown under certain heterotrophic conditions with nitrogen starvation (Miao and Wu, Bioresource Technology 97:841-846 (2006)). Other Chlorella species, including Chlorella emersonii, Chlorella sorokiniana and Chlorella minutissima have been described to have accumulated up to 63% oil by DCW when grown in stirred tank bioreactors under low-nitrogen media conditions (Illman et al., Enzyme and Microbial Technology 27:631-635 (2000). Still higher percent lipid by DCW has been reported, including 70% lipid in Dumaliella tertiolecta cultures grown in increased NaCl conditions (Takagi et al., Journal of Bioscience and Bioengineering 101(3): 223-226 (2006)) and 75% lipid in Botryococcus braunii cultures (Banerjee et al., Critical Reviews in Biotechnology 22(3): 245-279 (2002)).

Heterotrophic growth results in relatively low chlorophyll content (as compared to phototrophic systems such as open ponds or closed photobioreactor systems). The reduced chlorophyll content found in heterotrophically grown microalgae (e.g., Chlorella) also reduces the green color in the biomass as compared to phototrophically grown microalgae. Thus, the reduced chlorophyll content avoids an often undesired green coloring associated with cosmetic products containing phototrophically grown microalgae and allows for the incorporation or an increased incorporation of algal biomass into a cosmetic product. In at least one embodiment, the cosmetic product contains heterotrophically grown microalgae of reduced chlorophyll content compared to phototrophically grown microalgae.

Oil rich microalgal biomass generated by the culture methods described herein and useful in accordance with the present invention comprises at least 10% microalgal oil by DCW. In some embodiments, the microalgal biomass comprises at least 15%, 25%, 50%, 75% or at least 90% microalgal oil by DCW.

The microalgal oil of the biomass described herein (or extracted from the biomass) can comprise glycerolipids with one or more distinct fatty acid ester side chains. Glycerolipids are comprised of a glycerol molecule esterified to one, two, or three fatty acid molecules, which can be of varying lengths and have varying degrees of saturation. Specific blends of algal oil can be prepared either within a single species of algae, or by mixing together the biomass (or algal oil) from two or more species of microalgae.

Thus, the oil composition, i.e., the properties and proportions of the fatty acid constituents of the glycerolipids, can also be manipulated by combining biomass (or oil) from at least two distinct species of microalgae. In some embodiments, at least two of the distinct species of microalgae have different glycerolipid profiles. The distinct species of microalgae can be cultured together or separately as described herein, preferably under heterotrophic conditions, to generate the respective oils. Different species of microalgae can contain different percentages of distinct fatty acid constituents in the cell's glycerolipids.

In some embodiments, the microalgal oil is primarily comprised of monounsaturated oil. In some cases, the algal oil is at least 20% monounsaturated oil by weight. In various embodiments, the algal oil is at least 25%, 50%, 75% or more monounsaturated oil by weight or by volume. In some embodiments, the monounsaturated oil is 18:1, 16:1, 14:1 or 12:1. In some embodiments, the microalgal oil comprises at least 10%, 20%, 25%, or 50% or more esterified oleic acid or esterified alpha-linolenic acid by weight of by volume. In at least one embodiment, the algal oil comprises less than 10%, less than 5%, less than 3%, less than 2%, or less than 1% by weight or by volume, or is substantially free of, esterified docosahexanoic acid (DHA (22:6)). For examples of production of high DHA-containing microalgae, such as in Crypthecodinium cohnii, see U.S. Pat. Nos. 7,252,979, 6,812,009 and 6,372,460.

Microalgal biomass (and oil extracted therefrom), can also include other constituents produced by the microalgae, or incorporated into the biomass from the culture medium. These other constituents can be present in varying amounts depending on the culture conditions used and the species of microalgae (and, if applicable, the extraction method used to recover microalgal oil from the biomass). The other constituents can include, without limitation, phospholipids (e.g., algal lecithin), carbohydrates, soluble and insoluble fiber, glycoproteins, phytosterols (e.g., β-sitosterol, campesterol, stigmasterol, ergosterol, and brassicasterol), tocopherols, tocotrienols, carotenoids (e.g., α-carotene, β-carotene, and lycopene), xanthophylls (e.g., lutein, zeaxanthin, α-cryptoxanthin, and β-cryptoxanthin), proteins, polysaccharides (e.g., arabinose, mannose, galactose, 6-methyl galactose and glucose) and various organic or inorganic compounds (e.g., selenium). Microalgal sterols may have anti-inflammatory, anti-matrix-breakdown, and improvement of skin barrier effects when incorporated into a skincare product.

In some cases, the biomass comprises at least 10 ppm selenium. In some cases, the biomass comprises at least 25% w/w algal polysaccharide. In some cases, the biomass comprises at least 15% w/w algal glycoprotein. In some cases, the biomass comprises between 0-115 mcg/g total carotenoids. In some cases, the biomass comprises at least 0.5% algal phospholipids. In some cases, the oil derived from the algal biomass contains at least 0.10 mg/g total tocotrienols. In some cases, the oil derived from the algal biomass contains between 0.125 mg/g to 0.35 mg/g total tocotrienols. In some cases, the oil derived from the algal biomass contains at least 5.0 mg/100 g total tocopherols. In some cases, the oil derived from the algal biomass contains between 5.0 mg/100 g to 10 mg/100 g tocopherols.

In one aspect, the present invention is directed to a method of preparing algal oil by harvesting algal oil from an algal biomass comprising at least 15% oil by dry weight under GMP conditions, in which the algal oil is greater than 50% 18:1 lipid. In some cases, the algal biomass comprises a mixture of at least two distinct species of microalgae. In some cases, at least two of the distinct species of microalgae have been separately cultured. In at least one embodiment, at least two of the distinct species of microalgae have different glycerolipid profiles. In some cases, the algal biomass is derived from algae grown heterotrophically. In some cases, all of the at least two distinct species of microalgae contain at least 15% oil by dry weight.

oil can be separated from lysed biomass. The algal biomass remaining after oil extraction is referred to as delipidated meal. Delipidated meal contains less oil by dry weight or volume than the microalgae contained before extraction. Typically 50-90% of oil is extracted so that delipidated meal contains, for example, 10-50% of the oil content of biomass before extraction. However, the biomass still has a high nutrient value in content of protein and other constituents discussed above. Thus, the delipidated meal can be used in animal feed or in human food applications.

In some embodiments, the algal oil is at least 50% w/w oleic acid and contains less than 5% DHA. In some embodiments of the method, the algal oil is at least 50% w/w oleic acid and contains less than 0.5% DHA. In some embodiments of the method, the algal oil is at least 50% w/w oleic acid and contains less than 5% glycerolipid containing carbon chain length greater than 18. In some cases, the algal cells from which the algal oil is obtained comprise a mixture of cells from at least two distinct species of microalgae. In some cases, at least two of the distinct species of microalgae have been separately cultured. In at least one embodiment, at least two of the distinct species of microalgae have different glycerolipid profiles. In some cases, the algal cells are cultured under heterotrophic conditions. In some cases, all of the at least two distinct species of microalgae contain at least 10%, or at least 15% oil by dry weight.

In one aspect, provided is an algal oil containing at least 50% monounsaturated oil and containing less than 1% DHA prepared under GMP conditions. In some cases, the monounsaturated oil is 18:1 lipid. In some cases, the algal oil is packaged in a capsule for delivery of a unit dose of oil. In some cases, the algal oil is derived from a mixture of at least two distinct species of microalgae. In some cases, at least two of the distinct species of microalgae have been separately cultured. In at least one embodiment, at least two of the distinct species of microalgae have different glycerolipid profiles. In some cases, the algal oil is derived from algal cells cultured under heterotrophic conditions.

In one aspect, provided is an oil comprising greater than 60% 18:1, and at least 0.20 mg/g tocotrienol.

In one aspect, provided is a fatty acid alkyl ester composition comprising greater than 60% 18:1 ester, and at least 0.20 mg/g tocotrienol.

In one aspect, the algal oil is prepared from concentrated, washed microalgal biomass by extraction. The cells in the biomass are lysed prior to extraction. Optionally, the microbial biomass may also be dried (oven dried, lyophilized, etc.) prior to lysis (cell disruption). Alternatively, cells can be lysed without separation from some or all of the fermentation broth when the fermentation is complete. For example, the cells can be at a ratio of less than 1:1 v:v cells to extracellular liquid when the cells are lysed.

Microalgae containing lipids can be lysed to produce a lysate. As detailed herein, the step of lysing a microorganism (also referred to as cell lysis) can be achieved by any convenient means, including heat-induced lysis, adding a base, adding an acid, using enzymes such as proteases and polysaccharide degradation enzymes such as amylases, using ultrasound, mechanical pressure-based lysis, and lysis using osmotic shock. Each of these methods for lysing a microorganism can be used as a single method or in combination simultaneously or sequentially. The extent of cell disruption can be observed by microscopic analysis. Using one or more of the methods above, typically more than 70% cell breakage is observed. Preferably, cell breakage is more than 80%, more preferably more than 90% and most preferred about 100%.

Lipids and oils generated by the microalgae in accordance with the present invention can be recovered by extraction. In some cases, extraction can be performed using an organic solvent or an oil, or can be performed using a solventless-extraction procedure.

For organic solvent extraction of the microalgal oil, the preferred organic solvent is hexane. Typically, the organic solvent is added directly to the lysate without prior separation of the lysate components. In one embodiment, the lysate generated by one or more of the methods described above is contacted with an organic solvent for a period of time sufficient to allow the lipid components to form a solution with the organic solvent. In some cases, the solution can then be further refined to recover specific desired lipid components. The mixture can then be filtered and the hexane removed by, for example, rotoevaporation. Hexane extraction methods are well known in the art. See, e.g., Frenz et al., Enzyme Microb. Technol., 11:717 (1989).

Miao and Wu describe a protocol of the recovery of microalgal lipid from a culture of Chlorella protothecoides in which the cells were harvested by centrifugation, washed with distilled water and dried by freeze drying. The resulting cell powder was pulverized in a mortar and then extracted with n-hexane. Miao and Wu, Biosource Technology 97:841-846 (2006).

In some cases, microalgal oils can be extracted using liquefaction (see for example Sawayama et al., Biomass and Bioenergy 17:33-39 (1999) and Inoue et al., Biomass Bioenergy 6(4):269-274 (1993)); oil liquefaction (see for example Minowa et al., Fuel 74(12):1735-1738 (1995)); or supercritical CO₂ extraction (see for example Mendes et al., Inorganica Chimica Acta 356:328-334 (2003)).

Oil extraction includes the addition of an oil directly to a lysate without prior separation of the lysate components. After addition of the oil, the lysate separates either of its own accord or as a result of centrifugation or the like into different layers. The layers can include in order of decreasing density: a pellet of heavy solids, an aqueous phase, an emulsion phase, and an oil phase. The emulsion phase is an emulsion of lipids and aqueous phase. Depending on the percentage of oil added with respect to the lysate (w/w or v/v), the force of centrifugation if any, volume of aqueous media and other factors, either or both of the emulsion and oil phases can be present. Incubation or treatment of the cell lysate or the emulsion phase with the oil is performed for a time sufficient to allow the lipid produced by the microorganism to become solubilized in the oil to form a heterogeneous mixture.

Lipids can also be extracted from a lysate via a solventless extraction procedure without substantial or any use of organic solvents or oils by cooling the lysate. Sonication can also be used, particularly if the temperature is between room temperature and 65° C. Such a lysate on centrifugation or settling can be separated into layers, one of which is an aqueous:lipid layer. Other layers can include a solid pellet, an aqueous layer, and a lipid layer. Lipid can be extracted from the emulsion layer by freeze thawing or otherwise cooling the emulsion. In such methods, it is not necessary to add any organic solvent or oil. If any solvent or oil is added, it can be below 5% v/v or w/w of the lysate.

The oils produced according to the above methods in some cases are made using a microalgal host cell. As described above, the microalga can be, without limitation, fall in the classification of Chlorophyta, Trebouxiophyceae, Chlorellales, Chlorellaceae, or Chlorophyceae. It has been found that microalgae of Trebouxiophyceae can be distinguished from vegetable oils based on their sterol profiles. Oil produced by Chlorella protothecoides was found to produce sterols that appeared to be brassicasterol, ergosterol, campesterol, stigmasterol, and β-sitosterol, when detected by GC-MS. However, it is believed that all sterols produced by Chlorella have C24β stereochemistry. Thus, it is believed that the molecules detected as campesterol, stigmasterol, and β-sitosterol, are actually 22,23-dihydrobrassicasterol, proferasterol and clionasterol, respectively. Thus, the oils produced by the microalgae described above can be distinguished from plant oils by the presence of sterols with C24β stereochemistry and the absence of C24α stereochemistry in the sterols present. For example, the oils produced may contain 22,23-dihydrobrassicasterol while lacking campesterol; contain clionasterol, while lacking in β-sitosterol, and/or contain poriferasterol while lacking stigmasterol. Alternately, or in addition, the oils may contain significant amounts of Δ⁷-poriferasterol.

In one embodiment, the oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

Sterol profile analysis is a particularly well-known method for determining the biological source of organic matter. Campesterol, b-sitosterol, and stigamsterol are common plant sterols, with b-sitosterol being a principle plant sterol. For example, b-sitosterol was found to be in greatest abundance in an analysis of certain seed oils, approximately 64% in corn, 29% in rapeseed, 64% in sunflower, 74% in cottonseed, 26% in soybean, and 79% in olive oil (Gul et al. J. Cell and Molecular Biology 5:71-79, 2006).

Oils isolated from Prototheca moriformis strain UTEX1435 were separately clarified (CL), refined and bleached (RB), or refined, bleached and deodorized (RBD) and were tested for sterol content according to the procedure described in JAOCS vol. 60, no. 8, August 1983. Results of the analysis are shown below (units in mg/100 g):

| | | | | | Refined, |
|---|---|---|---|---|---|
| | | | | Refined & | bleached, & |
| | Sterol | Crude | Clarified | bleached | deodorized |
| 1 | Ergosterol | 384 (56%) | 398 (55%) | 293 (50%) | 302 (50%) |
| 2 | 5,22-cholestadien-24- | 14.6 (2.1%) | 18.8 (2.6%) | 14 (2.4%) | 15.2 (2.5%) |
| | methyl-3-ol | | | | |
| | (Brassicasterol) | | | | |
| 3 | 24-methylcholest-5-en- | 10.7 (1.6%) | 11.9 (1.6%) | 10.9 (1.8%) | 10.8 (1.8%) |
| | 3-ol (Campersterol or | | | | |
| | 22,23- | | | | |
| | dihydrobrassicasterol) | | | | |
| 4 | 5,22-cholestadien-24- | 57.7 (8.4%) | 59.2 (8.2%) | 46.8 (7.9%) | 49.9 (8.3%) |
| | ethyl-3-ol (Stigmaserol | | | | |
| | or poriferasterol) | | | | |
| 5 | 24-ethylcholest-5-en-3- | 9.64 (1.4%) | 9.92 (1.4%) | 9.26 (1.6%) | 10.2 (1.7%) |
| | ol (β-Sitosterol or | | | | |
| | clionasterol) | | | | |
| 6 | Other sterols | 209 | 221 | 216 | 213 |
| | Total sterols | 685.64 | 718.82 | 589.96 | 601.1 |

These results show three striking features. First, ergosterol was found to be the most abundant of all the sterols, accounting for about 50% or more of the total sterols. The amount of ergosterol is greater than that of campesterol, β-sitosterol, and stigamsterol combined. Ergosterol is steroid commonly found in fungus and not commonly found in plants, and its presence particularly in significant amounts serves as a useful marker for non-plant oils. Secondly, the oil was found to contain brassicasterol. With the exception of rapeseed oil, brassicasterol is not commonly found in plant based oils. Thirdly, less than 2% β-sitosterol was found to be present. β-sitosterol is a prominent plant sterol not commonly found in microalgae, and its presence particularly in significant amounts serves as a useful marker for oils of plant origin. In summary, Prototheca moriformis strain UTEX1435 has been found to contain both significant amounts of ergosterol and only trace amounts of β-sitosterol as a percentage of total sterol content. Accordingly, the ratio of ergosterol: β-sitosterol or in combination with the presence of brassicasterol can be used to distinguish this oil from plant oils.

In some embodiments, the oil content of an oil provided herein contains, as a percentage of total sterols, less than 20%, 15%, 10%, 5%, 4%, 3%, 2%, or 1% β-sitosterol. In other embodiments the oil is free from β-sitosterol.

In some embodiments, the oil is free from one or more of β-sitosterol, campesterol, or stigmasterol. In some embodiments the oil is free from β-sitosterol, campesterol, and stigmasterol. In some embodiments the oil is free from campesterol. In some embodiments the oil is free from stigmasterol.

In some embodiments, the oil content of an oil provided herein comprises, as a percentage of total sterols, less than 20%, 15%, 10%, 5%, 4%, 3%, 2%, or 1% 24-ethylcholest-5-en-3-ol. In some embodiments, the 24-ethylcholest-5-en-3-ol is clionasterol. In some embodiments, the oil content of an oil provided herein comprises, as a percentage of total sterols, at least 1%, 2%, 3%, 4%, 5%, 6%, 7%, 8%, 9%, or 10% clionasterol.

In some embodiments, the oil content of an oil provided herein contains, as a percentage of total sterols, less than 20%, 15%, 10%, 5%, 4%, 3%, 2%, or 1% 24-methylcholest-5-en-3-ol. In some embodiments, the 24-methylcholest-5-en-3-ol is 22,23-dihydrobrassicasterol. In some embodiments, the oil content of an oil provided herein comprises, as a percentage of total sterols, at least 1%, 2%, 3%, 4%, 5%, 6%, 7%, 8%, 9%, or 10% 22,23-dihydrobrassicasterol.

In some embodiments, the oil content of an oil provided herein contains, as a percentage of total sterols, less than 20%, 15%, 10%, 5%, 4%, 3%, 2%, or 1% 5,22-cholestadien-24-ethyl-3-ol. In some embodiments, the 5,22-cholestadien-24-ethyl-3-ol is poriferasterol. In some embodiments, the oil content of an oil provided herein comprises, as a percentage of total sterols, at least 1%, 2%, 3%, 4%, 5%, 6%, 7%, 8%, 9%, or 10% poriferasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4% or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

In some embodiments, the oil content contains, as a percentage of total sterols, at least 5%, 10%, 20%, 25%, 35%, 40%, 45%, 50%, 55%, 60%, or 65% ergosterol and less than 20%, 15%, 10%, 5%, 4%, 3%, 2%, or 1% β-sitosterol. In some embodiments, the oil content contains, as a percentage of total sterols, at least 25% ergosterol and less than 5% β-sitosterol. In some embodiments, the oil content further comprises brassicasterol.

### EXAMPLES

### Example 1

Strains were prepared and grown heterotrophically as described above and in WO2008/151149, WO2010/063031, WO2010/045368, WO2010/063032, WO2011/150411, WO2013/158938, 61/923,327 filed Jan. 3, 2014, PCT/US2014/037898 filed May 13, 2014, and in U.S. Pat. No. 8,557,249. Sample A refers to oil from Chlorella (Auxeochlorella) protothecoides cells (UTEX 250). Samples B-F are oil isolated from various strains originating from Prototheca moriformis (UTEX 1435) that were prepared and cultured to achieve the indicated fatty acid profile. UTEX 250 and 1435 are available from the University of Texas at Austin Culture Collection of Algae.

TABLE I
Oil properties

| | | Sample | | | | | | |
|---|---|---|---|---|---|---|---|---|
| Assay | | A (UTEX | B (high | C | D (high | E | F (low poly- | |
| Fatty Acid Profile | Units | 250) | C10-C12) | (laurate) | myristic) | (SOS) | unsaturates) | G (Oleic) |
| C8:0 | % | 0.00 | 1.02 | 0.35 | 0.00 | 0.00 | 0.00 | 0.00 |
| C10:0 | % | 0.08 | 40.45 | 18.18 | 0.04 | 0.03 | 0.03 | 0.01 |
| C12:0 | % | 0.22 | 45.00 | 45.92 | 0.89 | 0.19 | 0.06 | 0.03 |
| C14:0 | % | 1.29 | 4.00 | 12.92 | 56.94 | 0.47 | 0.35 | 0.41 |
| C16:0 | % | 17.44 | 2.33 | 6.34 | 14.98 | 3.03 | 3.29 | 3.31 |
| C18:0 | % | 1.66 | 0.27 | 0.51 | 0.68 | 56.75 | 2.87 | 2.22 |
| C18:1 | % | 59.12 | 4.24 | 10.12 | 20.51 | 33.90 | 89.94 | 86.17 |
| C18:2 | % | 15.17 | 1.62 | 3.32 | 4.26 | 1.94 | 1.03 | 5.50 |
| C18:3 ALPHA | % | 2.01 | 0.27 | 0.38 | 0.23 | 0.16 | 0.15 | 0.24 |
| C20:0 | % | 0.25 | 0.02 | 0.06 | 0.06 | 1.65 | 0.25 | 0.26 |
| DROPPING | ° C. | 10.5 | 22.2 | 27.2 | | | 2 | 0.3 |
| MELTING POINT | | | | | | | | |
| (METTLER) AOCS | | | | | | | | |
| Cc 18-80 | | | | | | | | |
| CLOUD | ° C. | | 12 | 17 | 29 | | −18 | −19 |
| POINT D97 | | | | | | | | |
| POUR | ° C. | | 10 | 15 | 27 | | −20 | −21 |
| POINT D97 | | | | | | | | |
| IODINE VALUE | unit | 85.6 | 8.8 | 18.7 | 27.7 | | 81.6 | 85.6 |
| OSI RANCIMAT | hours | | 68.72 | 46.8 | 37.56 | | 57.6 | 19.35 |
| (110° C.) AOCS Cd | | | | | | | | |
| 12b-92 | | | | | | | | |
| SMOKE POINT | ° C. | | | | 150 | | 248 | 248 |
| AOCS Cc 9a-48 | | | | | | | | |
| SAPONIFICATION | mg | | | 239.2 | | | | |
| VALUE AOCS Cd | KOH/g | | | | | | | |
| 3-25 | | | | | | | | |
| ALPHA | mg/100 g | 12.7 | — | | 0.22 | | — | — |
| TOCOPHEROL | | | | | | | | |
| B-SITOSTEROL | mg/100 g | 56.3 | — | | 6.51 | | 26.4 | 3.81 |
| BETA | mg/100 g | — | — | | — | | — | — |
| TOCOPHEROL | | | | | | | | |
| BRASSICASTEROL | mg/100 g | 131 | — | | — | | — | — |
| CAMPESTEROL | mg/100 g | 16.8 | 11.9 | | 6.29 | 3.72 | 8.03 | 8.08 |
| CHOLESTEROL | mg/100 g | — | — | | — | | — | — |
| DELTA | mg/100 g | 5.47 | 0.76 | | 0.28 | 1.48 | — | 0.81 |
| TOCOPHEROL | | | | | | | | |
| ERGOSTEROL | mg/100 g | 130 | 59.2 | | 174 | 54.8 | 174 | 92 |
| GAMMA | mg/100 g | 2.25 | — | | 0.28 | 0.83 | 0.57 | 0.12 |
| TOCOPHEROL | | | | | | | | |
| STIGMASTEROL | mg/100 g | 18.7 | 6.19 | | 16.3 | 13.3 | 15.7 | 11.6 |
| OTHER STEROLS | mg/100 g | 279 | 111 | | 151 | 139 | 98.3 | 130 |
| ALPHA | mg/g | 0.11 | | | 0.18 | 0.17 | | |
| TOCOTRIENOL | | | | | | | | |
| BETA | mg/g | 0.02 | | | 0.04 | <0.01 | | |
| TOCOTRIENOL | | | | | | | | |
| DELTA | mg/g | 0.06 | | | <0.01 | <0.01 | | |
| TOCOTRIENOL | | | | | | | | |
| GAMMA | mg/g | 0.02 | | | 0.03 | 0.07 | | |
| TOCOTRIENOL | | | | | | | | |
| TOTAL | mg/g | 0.21 | | | 0.25 | 0.24 | | |
| TOCOTRIENOLS | | | | | | | | |

### Example 2

Algal oils prepared according to the methods described above and having profiles similar to samples B (C10/C12) and C (C12/C14) in Table 1 were derivatized as fatty acid methyl esters. The esters were found to have the following properties when subjected to tests indicated in the table below. Methyl esters having the sample B profile were found to have a superior Kauri-Butanol value.

TABLE II
Methyl esters

| Test | Unit | Method | C10/C12 | C12/C14 |
|---|---|---|---|---|
| Kauri-Butanol | ° C. | ASTM D1133 | 79.0 | 71 |
| Value | | | | |
| Flash Point | ° C. | ASTM D92 | 116 | 125 |
| (COC) | | | | |
| Kinematic | cSt | ASTM D445 | 2.202 | 2.786 |
| Viscosity, 40° C. | | | | |
| Residue on | % | ASTM D1353 | 0.27 | 0.62 |
| Evaporation | | | | |

1. A method for preparing a surfactant, the method comprising:
a) providing a triglyceride oil produced by a microalgae; and
b) subjecting the oil to one or more chemical reactions to produce a surfactant.

2. The method of claim 1, wherein the surfactant is an anionic surfactant.

3. The method of claim 2, wherein the surfactant is a sulfonate having a —SO₃M moiety wherein M is an alkali metal, a sulfate having a —OSO₃M moiety wherein M is an alkali metal, or wherein the surfactant comprises an amino acid moiety.

4-24. (canceled)

25. The method of claim 1, wherein the surfactant is a nonionic surfactant.

26. The method of claim 25, wherein the nonionic surfactant comprises one or more of ethylene oxide or propylene oxide moieties or combinations thereof and an OH or SH moiety, or wherein the nonionic surfactant is an alkyl polyglycoside, or wherein the nonionic surfactant comprises a glycerin moiety.

27-33. (canceled)

34. The method of claim 1, wherein the surfactant is an amphoteric surfactant.

35. The method of claim 34, wherein the amphoteric surfactant is a betaine or an amine oxide.

36-40. (canceled)

41. The method of claim 1, wherein the surfactant is a cationic surfactant.

42. The method of claim 41, wherein the surfactant comprises a quaternary ammonium salt, or is an ester quat.

43-46. (canceled)

47. The method of claim 1, wherein the microalgae is of the genus Prototheca, Auxenochlorella, Chlorella, or Parachlorella.

48. The method of claim 1, wherein the microalgae is of the species Prototheca moriformis or Chlorella (Auxeochlorella) protothecoides.

49. (canceled)

50. The method of claim 1, wherein the microalgae is Chlorella (Auxeochlorella) protothecoides and the oil has a fatty acid profile of greater than 15% C16:0 and greater than 55% 18:1.

51. The method of claim 1, wherein the oil has a fatty acid profile of greater than 50%, 60%, 70%, or 80% combined C10:0 and C12:0.

52. The method of claim 1, wherein the oil has a fatty acid profile of greater than 60% C10:0 and C12:0 and greater than 10% C14:0.

53. The method of claim 1, wherein the oil has a fatty acid profile of greater than 40%, 45%, or 50% C14:0.

54. The method of claim 1, wherein the oil has a fatty acid profile of greater than 85% C18:1 and less than 3% polyunsaturates.

55. The method of claim 1, wherein the oil has a fatty acid profile of at least 70% SOS and no more than 4% trisaturates.

56. The method of claim 1, wherein the oil has a fatty acid profile of greater than 50% C18:0 and greater than 30% C18:1.

57. A composition comprising the surfactant of claim 1.

58. The composition of claim 57, wherein the composition is a cleaner, a metal working fluid, a pesticide, an agricultural adjuvant, a wetting agent, a dispersant, a buffer, a compatibility agent, a crop oil, a defoaming agent, a spreader, a detergent, a fabric softener, a cosmetic, a shampoo, a hair conditioner, a toothpaste, a sanitizer, an emulsion, an ink, an adhesive, a paint, a lubricant, a solvent, a coating, a sealant, an elastomer, a polymer, a pharmaceutical drug, a vaccine adjuvant, or a wellbore fluid.

59-60. (canceled)
